(19) United States
(12) Patent Application Publication
(10) Pub. No.: US 2004/0259809 A1
(43) Pub. Date: Dec. 23, 2004

(54) Pharmaceutical compositions including an antihistamine and a stimulant and method of use thereof

(75) Inventor: Gilbert Rene Gonzales, New York, NY (US)

Correspondence Address: WOOD, HERRON & EVANS, LLP, 2700 CAREW TOWER, 441 VINE STREET, CINCINNATI, OH, 45202, US

(73) Assignee: PediaMed Pharmaceuticals, Inc., Exton, PA

(21) Appl. No.: 10/463,556

(22) Filed: Jun. 17, 2003

(51) Int. Cl. A61K 31/70; A61K 31/542; A61K 31/538; A61K 31/496; A61K 31/495; A61K 31/522; A61K 31/137

(52) U.S. Cl. 514/23; 514/224.8; 514/255.04; 514/263.32; 514/649; 514/230.2

(57) ABSTRACT

The present invention provides pharmaceutical compositions including a sedating antihistamine and a stimulant, and methods of use thereof. The stimulant reduces the sedation caused by the antihistamine, thereby allowing potent, but sedating, antihistamines to be used effectively.

### BACKGROUND OF THE INVENTION

I. Field of the Invention

The present invention relates to pharmaceutical compositions including an antihistamine and a stimulant, and to methods of use to treat histamine-mediated symptoms.

II. Description of the Prior Art

Histamine is a biologically active amine found in many tissues and is frequently released locally to induce complex physiologic and pathologic effects through multiple histamine receptor subtypes. Three different receptor sites for histamine have been recognized, and are designated H₁, H₂, and H₃. Histamine is recognized as an important mediator of immediate allergic and inflammatory reactions. Generally, upon exposure of the body to a variety of immunogenic stimuli, such as pollen, dust, toxins, and the like, histamine is released into the blood circulation to induce an allergic-type response in the body. Particularly, histamine stored in mast cells and basophil cells is often released upon sensitization by IgE antibodies attached to the cell surface membranes. Histamine released by this mechanism is a mediator in immediate (type 1) allergic reactions. In addition, by a negative feedback control mechanism mediated by H₂ receptors, histamine appears to modulate its own release and that of other mediators from sensitized mast cells in some tissues. In addition to inducing allergic response, histamine plays many other roles, including a role in acute inflammatory responses.

Histamine is also known to exert powerful effects on smooth and cardiac muscle, on certain endothelia and nerve cells, and on secretory cells of the stomach. Particularly, histamine causes contraction of intestinal smooth muscle. Large doses of histamine may even cause diarrhea. Histamine has long been recognized as a powerful stimulant of gastric acid secretion and, to a lesser extent, of gastric pepsin and intrinsic factor production. In the central nervous system, histamine stimulates sensory nerve endings, especially those mediating pain and itching. This H₁ mediated effect is an important component of the urticarial response and reactions to insect and nettle stings. Pre-synaptic H₃ receptors play important roles in modulating neurotransmitter release from peripheral nerve endings. In the cardiovascular system, histamine causes a decrease in systolic and diastolic blood pressure and an increase in heart rate. Particularly, the acute blood pressure changes are caused by the direct vasodilator action of histamine on arterial and capillary sphincters and the increase in heart rate involves both stimulatory actions of histamine on the heart, and a reflex tachycardia. Histamine induced edema results from the action of the histamine on the H₁ receptors in the blood vessels. The effect is associated with the separation of endothelial cells, which permits the transudation of fluid and molecules as large as small proteins into the perivascular tissue. This effect is responsible for the urticaria (hives) that signals the release of histamine in the skin.

Histamine also causes bronchial constriction mediated by H₁ receptors. Thus, humans suffering from asthma are very sensitive to histamine. The bronchial constriction induced in these patients probably represents a hyperactive neural response, since such patients also respond excessively to many other stimuli, and their response to histamine can be blocked by autonomic blocking drugs such as ganglionic blocking agents as well as H₁ receptor antagonists. Sensitivity to histamine, however, varies greatly among mammals.

Thus, the release of histamine in the body causes many adverse physiological and pathological responses or effects in mammals and is induced, or stimulated, by common, everyday environmental factors and/or allergies to those factors. There is, therefore, a need to reduce or alleviate these effects and/or prevent their induction by histamine.

The effects of histamine released in the body may be treated or addressed in several ways. Histamine receptor antagonists, such as epinephrine, are generally referred to as antihistamines and administered to counter the effects of histamine. But epinephrine antagonists, and derivatives thereof, act at receptors other than those activated by histamine and are, therefore, not highly effective in alleviating or preventing a histamine-mediated response. Other antihistamines, such as diphenhydramine, act directly on the histamine receptor(s) to reduce or prevent histamine-mediated responses. For example, medications containing diphenhydramine, or similarly structured compounds, are available to competitively antagonize many of the actions of histamine.

Over the years, a number of antihistamines (histamine antagonists) have been developed and are generally divided into two groups: the first-generation antihistamines and second-generation antihistamines. The two groups are distinguished by the relatively strong sedative effects of the first-generation antihistamines. First-generation antihistamines are capable of crossing the blood-brain barrier and entering the central nervous system. Common first-generation antihistamines include compounds such as diphenhydramine, brompheneramine, hydrazine, and chloropheneramine, all of which are able to cross the blood-brain barrier and are sedating in effect. The intensity of sedation varies among the antihistamines depending upon the chemical structure. For some antihistamines, the sedation is so prominent that they are useful as “sleep aids” and unsuitable for daytime use. Sedation, however, also renders these antihistamines potentially dangerous depending upon the person's activity after taking the antihistamine. For example, driving after having taken a recommended dose of a first-generation antihistamine may result in falling asleep at the wheel consequentially presenting risks of injury, property damage, etc. Accordingly, sedation may interfere with everyday activity and is, therefore, typically an undesirable effect of the first-generation antihistamines.

The sedative effect of first-generation antihistamines has prompted the development of newer, second-generation antihistamines that cause significantly reduced sedation or no sedation at all. However, these second-generation antihistamines are generally less effective than the first-generation antihistamines for treating congestion in the upper airways, viral and allergic rhinitis, generalized allergy symptoms, and other symptoms associated and mediated by receptor-bound histamine. Therefore, second-generation antihistamines are required in larger doses to be as therapeutically effective as the first generation antihistamines. Higher dosages render medications containing second-generation antihistamines not only more costly to administer, but also more likely to lead to a higher incidence of undesirable side effects and other problems associated with use. Accordingly, with the exception of the sedative effects, the first-generation antihistamines would be more useful in general than the second-generation antihistamines in terms of efficacy and potency as antihistaminics. Thus, there is a need to provide pharmaceutical compositions comprising an antihistamine, which may be administered in therapeutically effective dose, while minimizing undesirable sides effects, such as sedation.

### SUMMARY OF THE INVENTION

The present invention addresses the weaknesses and drawbacks of prior art antihistamine-containing compositions by providing pharmaceutical compositions that may be administered in effective dosages for treating histamine-mediated symptoms with a reduced or minimal corresponding sedative effect. To this end, the pharmaceutical compositions include at least one antihistamine and at least one stimulant. The most potent antihistamines are generally sedating in nature and the sedation is reduced or alleviated with the stimulant. The compositions are useful for treating allergic reactions and other histamine-mediated symptoms, as well as for providing other physiological effects including, for example, anticholinergic effects, analgesic effects, analgesic adjuvant effects, soporific effects, anti-secretory effects, and combination effects thereof. By combining a potent, effective antihistamine with an effective, anti-sedating stimulant, the compositions of the present invention may be administered more safely than prior art antihistamine-containing medications utilized for the same purpose. The present invention also provides methods of use for the pharmaceutical compositions.

Antihistamines suitable for the compositions include, without limitation, diphenhydramine, cyproheptadine hydrochloride, brompheneramine, hydroxyzine, chloropheniramine, pyrilamine maleate, pyrilamine tannate, acepromazine, aceprometazine, alimemazine, alimemazine tartrate, amoxydramine camsilate, antazoline chlorhydrate, antazoline mesilate, antazoline phosphate, astemizole, azatadine dimaleate, azelastine hydrochloride, bamipine hydrochloride, benactyzine hydrochloride, bretylium tosilate, bromazine hydrochloride, brompheniramine maleate, buclizine dihydrochloride, bufexamac, carbinoxamine maleate acid, cetiedil citrate, cetirizine dihydrochloride, chlorcyclizine hydrochloride, chlorphenamine maleate, chlorphenoxamine hydrochloride, chlorprothixene hydrochloride, cinnarizine, clemastine fumarate, clemizole hexachlorophenate, clemizole penicilline, clemizole undecylenate, clocinizine dihydrochloride, clofedanol, clofenetamine hydrochloride, cyclizine hydrochloride, dexchlorpheniramine maleate, di(acefylline)diphenhydramine, difencloxazine, dimelazine hydrochloride, dimenhydrinate, dimethoxanate hydrochloride, cimetotiazine mesilate, diphenhydramine hydrochloride, diphenhydramine mesilate, diphenylpyraline hydrochloride, diproqualone camsilate, dixyrazine, doxylamine succinate, eprozinol dihydrochloride, etodroxizine dimaleate, etybenzatropine bromhydrate, etybenzatropine hydrochloride, etymemazine hydrochloride, fenethazine hydrochloride, fenoxazoline hydrochloride, fenpentadiol, flunarizine hydrochloride, flupentixol decanoate, flupentixol dihydrochloride, histapyrrodine hydrochloride, hydroxyzine dihydrochloride, hydroxyzine embonate, indoramine hydrochloride, isothipendyl hydrochloride, ketotifene fumarate, levocabastine hydrochloride, levomepromazine, levomepromazine hydrochloride, levomepromazine embonate, levomepromazine maleate, loratadine, maprotiline hydrochloride, maprotiline mesilate, maprotiline resinate, meclozine hydrochloride, mecysteine hydrochloride, medifoxamine fumarate, mefenidramium metilsulfate, mepyramine maleate, mequitazine, methaqualone, methdilazine hydrochloride, metixene hydrochloride, mizolastine, moxisylyte hydrochloride, niaprazine, orphenadrine hydrochloride, oxaflumazine disuccinate, oxatomide, oxolamine benzilate, oxolamine citrate, oxomemazine, oxomemazine hydrochloride, parathiazine teoclate, perimetazine, pheniramine maleate, phenoxybenzamine hydrochloride, phenyltoloxamine, phenyltoloxamine citrate, pimethixene, pipotiazine, pipretecol dihydrochloride, pizotifene malate, prednazoline, profenamine hydrochloride, promethazine, promethazine hydrochloride, promethazine embonate, promethazine polyvinylbenzene-metacrylate, propiomazine, terfenadine, thenalidine tartrate, thenyldiamine hydrochloride, thiazinamium metilsulfate, tripelennamine hydrochloride, triprolidine hydrochloride, and tymazoline hydrochloride, and combinations thereof.

The antihistamine is included in an amount, per dosage of the composition, sufficient to alleviate one or more histamine-mediated responses in a patient. Effective doses of the antihistamine will generally vary depending upon the antihistamine(s) administered. For example, diphenhydramine (Benedryl) may be effective if administered in an amount of at least about 6 mg per dose and advantageously in an amount ranging from about 6 mg to about 100 mg per dose. Cyproheptadine may be effective if administered in an amount of at least about 0.05 mg per dose, and advantageously in an amount ranging from about 0.5 mg to about 20 mg per dose.

The stimulant is utilized to reduce or alleviate the sedation caused by the antihistamine. For this purpose, suitable effective stimulants include, for example, amphetamine-class compounds, such as dextroamphetamine, and non-amphetamine class compounds, such as caffeine, pemoline, methylphenidate, modafinil, and their pharmaceutically acceptable salts or derivatives.

The stimulant is included in an amount, per dose of the composition, sufficient to effectively counter-act the sedative effect of the antihistamine. For example, caffeine or a pharmaceutically acceptable salt or derivative thereof, is generally effective if administered in an amount ranging from about 50 mg to about 500 mg per dose. Combinations of particular stimulants are also known to be effective. For example, a combination of amphetamines may also be effective. It should be appreciated by those of ordinary skill in the art that the effective dosage amounts of the antihistamine and stimulant will generally vary depending on patient profile, such as the age, size, gender, and medical history, and patient group for which the composition is be administered.

The present composition may further include active ingredients where the antihistamine is not diphenhydramine or the stimulant is not caffeine. Such actives include, without limitation, sympathomimetic agents, opioid agents, anti-tussive agents, anti-secretory agents, analgesic agents, and the like. The composition may be formulated for enteral and/or parenteral administration. For example, suitable orally administered formulations include a tablet, pill, or capsule, while suitable rectally administered formulations include a suppository. In one embodiment of the present invention, the composition includes at least one antihistamine and at least one caffeine-free stimulant for treating histamine-mediated responses. In another embodiment, the composition includes at least one diphenhydramine-free antihistamine and at least one stimulant. In another embodiment, the composition includes cyproheptadine as the antihistamine in combination with at least one stimulant.

By virtue of the foregoing, there is thus provided compositions and methods for alleviating, in a patient, symptoms and conditions mediated by histamine, such as allergic responses, that may be administered more safely than comparable prior art antihistamine-containing compositions. These and other advantages and benefits of the present invention shall be made apparent from the accompanying detailed description thereof.

### DETAILED DESCRIPTION OF EXEMPLARY EMBODIMENTS

The present invention provides pharmaceutical compositions, and methods of use thereof, for treating histamine-mediated responses, particularly allergy and allergy-related symptoms, and providing beneficial physiological effects in a mammal, more safely than comparable medications of the prior art. The phrase “histamine-mediated response”, as used herein, is intended to refer to physiological responses or effects triggered, and often mediated, by the production, release, and binding of histamine to histamine-receptors in the body. Treatable histamine-mediated responses include a wide range of symptoms, including allergy symptoms related to a common cold or flu, such as congestion in the upper airways, cough, fever, runny nose, and the like, as well as hives, breakouts, itching, and swelling due to common allergens, such as pollen, dust, foods, and the like, or other external stimulants. “Histamine-mediated responses” also includes many non-allergy type symptoms, such as those discussed herein in the background section of the invention. The term “amount sufficient”, as used here, is intended to refer to an amount, of the ingredient to which it refers, capable of producing the physiological response for which the ingredient is known. For example, an amount sufficient of an antihistamine is an amount of the antihistamine capable of agonizing or antagonizing the effects of histamine in the body (an antihistaminic effect). Also, an amount sufficient of a stimulant would be that amount necessary to produce a physiological response in the central nervous system so as to accelerate nerve transmission and create a feeling of invigoration (stimulation) in the body. The term “pharmaceutically acceptable salt”, as used herein with reference to an antihistamine and a stimulant, is intended to refer to all salt forms of an active ingredient, such as the antihistamine and stimulant. For example, any acid, base, or halide counter-ion which forms a salt with the active and is biologically safe for mammalian consumption is suitable. Non-limiting examples include a hydrochloride, a citrate, a maleate, a fumarate, a succinate, a saccarate, an aspartate, a sulfate, an amine salt, an amino acid salt, and the like. Pharmaceutically acceptable salts of active ingredients are generally known to those of ordinary skill in the art. The term “derivative” as used herein, is intended to refer to compounds having the active chemical core structure with one or more inert structural modifications thereon. In addition, a derivative would include all such compounds, which do not change or alter the pharmacological mechanism of action and/or window of therapeutic efficacy of the core structure. For example, a “derivative” of diphenhydramine would include compounds having one or more methyl substitutions on the phenyl ring or aliphatic regions of the diphenhydramine core and which would not significantly differ in efficacy and mode of action. Many “derivative” compounds, for the antihistamine actives and stimulant actives, are known in the art and/or will be discovered and are contemplated herein. As such, the term “derivative” is used broadly herein, as appreciated by one of ordinary skill in the art.

The compositions of the present invention include at least one antihistamine and at least one stimulant and may be administered in a pharmaceutically acceptable formulation. The antihistamine is therapeutically effective to treat histamine-mediated responses, such as by providing relief from upper-respiratory congestion and viral and allergic rhinitis. Generally, the most potent antihistamines are sedative in nature by crossing the blood-brain barrier. The stimulant is provided, therefore, to alleviate such sedation. Thus, the stimulant addresses potential undesirable side effects typically caused by many sedating antihistamines, and in particular first-generation antihistamines that are otherwise proven therapeutically more effective than second-generation antihistamines. For example, the present invention would reduce or eliminate dangers related to driving while sedated, and related consequences such as personal injury, property damage, etc.

Suitable antihistamines include, without limitation, diphenhydramine, cyproheptadine hydrochloride, brompheneramine, hydroxyzine, chloropheniramine, pyrilamine maleate, pyrilamine tannate, acepromazine, aceprometazine, alimemazine, alimemazine tartrate, amoxydramine camsilate, antazoline chlorhydrate, antazoline mesilate, antazoline phosphate, astemizole, azatadine dimaleate, azelastine hydrochloride, bamipine hydrochloride, benactyzine hydrochloride, bretylium tosilate, bromazine hydrochloride, brompheniramine maleate, buclizine dihydrochloride, bufexamac, carbinoxamine maleate acid, cetiedil citrate, cetirizine dihydrochloride, chlorcyclizine hydrochloride, chlorphenamine maleate, chlorphenoxamine hydrochloride, chlorprothixene hydrochloride, cinnarizine, clemastine fumarate, clemizole hexachlorophenate, clemizole penicilline, clemizole undecylenate, clocinizine dihydrochloride, clofedanol, clofenetamine hydrochloride, cyclizine hydrochloride, dexchlorpheniramine maleate, di(acefylline)diphenhydramine, difencloxazine, dimelazine hydrochloride, dimenhydrinate, dimethoxanate hydrochloride, cimetotiazine mesilate, diphenhydramine hydrochloride, diphenhydramine mesilate, diphenylpyraline hydrochloride, diproqualone camsilate, dixyrazine, doxylamine succinate, eprozinol dihydrochloride, etodroxizine dimaleate, etybenzatropine bromhydrate, etybenzatropine hydrochloride, etymemazine hydrochloride, fenethazine hydrochloride, fenoxazoline hydrochloride, fenpentadiol, flunarizine hydrochloride, flupentixol decanoate, flupentixol dihydrochloride, histapyrrodine hydrochloride, hydroxyzine dihydrochloride, hydroxyzine embonate, indoramine hydrochloride, isothipendyl hydrochloride, ketotifene fumarate, levocabastine hydrochloride, levomepromazine, levomepromazine hydrochloride, levomepromazine embonate, levomepromazine maleate, loratadine, maprotiline hydrochloride, maprotiline mesilate, maprotiline resinate, meclozine hydrochloride, mecysteine hydrochloride, medifoxamine fumarate, mefenidramium metilsulfate, mepyramine maleate, mequitazine, methaqualone, methdilazine hydrochloride, metixene hydrochloride, mizolastine, moxisylyte hydrochloride, niaprazine, orphenadrine hydrochloride, oxaflumazine disuccinate, oxatomide, oxolamine benzilate, oxolamine citrate, oxomemazine, oxomemazine hydrochloride, parathiazine teoclate, perimetazine, pheniramine maleate, phenoxybenzamine hydrochloride, phenyltoloxamine, phenyltoloxamine citrate, pimethixene, pipotiazine, pipretecol dihydrochloride, pizotifene malate, prednazoline, profenamine hydrochloride, promethazine, promethazine hydrochloride, promethazine embonate, promethazine polyvinylbenzene-metacrylate, propiomazine, terfenadine, thenalidine tartrate, thenyldiamine hydrochloride, thiazinamium metilsulfate, tripelennamine hydrochloride, triprolidine hydrochloride, and tymazoline hydrochloride. Further, combinations of the above exemplary antihistamines may be included to provide the desired antihistaminic effects. The increased potency and effectiveness of first-generation antihistamines over the second generation, less sedating or non-sedating antihistamines may allow for the present compositions to have less quantities of the antihistamine and consequently be administered in smaller dosages or less frequently to the patient.

Many of the antihistamines above provide advantages and therapeutic benefits in addition to their proven antihistaminic effects. Thus, a composition including such an antihistamine, or a pharmaceutical acceptable salt or derivative thereof, may be effective for alleviating multiple allergy and cold-type symptoms, as well as for providing other effects, in both children and adults. Accordingly, the compositions of the present invention may have fewer actives and may be administered in smaller dosages while matching or exceeding the desired effects of comparable prior art medications lacking such an antihistamine.

Diphenhydramine, for example, has been shown to provide such additional benefits. Particularly, diphenhydramine has been shown to provide anticholinergic effects, analgesic effects, analgesic adjuvant effect, soporific effects, and anti-secretory effects (drying of mucous membranes). More specifically, diphenhydramine has been found to slow down or depress nerve activity by acting as an anticholinergic agent in the central nervous system. To this end, diphenhydramine may be administered to provide a sedative effect to cause drowsiness, thereby helping the person get rest and plenty of needed sleep. Large doses of the diphenhydramine are generally required to provide such a soporific effect. In addition, diphenhydramine has been clinically used as a drying agent or an anti-secretory agent to dry mucous membranes and prevent secretions therein. Diphenhydramine has also been shown to provide mild, local analgesic properties, and to enhance the effects of other analgesics in a synergistic manner as an analgesic adjuvant. For example, diphenhydramine has been shown to further the effects of opioids, chlomidene, acetaminophen, ibuprofen, aspirin, or other commonly administered painkillers. Thus, diphenhydramine provides additional benefits, which many other, commonly administered and effective antihistamines fail to provide. Accordingly, prior art medications having these antihistamines would require additional ingredients or increased dosages of specific ingredients to simultaneously provide the advantages of diphenhydramine.

The antihistamine should be included in an amount sufficient to effectively alleviate the histamine-mediated response, such as an allergy symptom or allergy-related symptom, and/or to provide other physiological effects, in a single dose of the composition. The potency of the antihistamine is generally dependent upon the particular antihistamine or combination thereof. For example, diphenhydramine is known to be effective in amounts as low as about 6 mg per dose or greater, depending upon the patient. More specifically, a diphenhydramine dose of about 6 mg to a child may be effective. Accordingly, the effective dosage may vary, as appreciated by one of ordinary skill in the art, depending upon many factors, such as age, weight, and gender of the patient, as well as prior medical history and present medical health. In one embodiment, the antihistamine is diphenhydramine, or a pharmaceutically acceptable salt or derivative thereof, in a weight ranging from about 6 mg to about 100 mg per dose of the composition. In another embodiment, diphenhydramine is present in a weight ranging from about 25 mg to about 75 mg per dose, and the composition is to be administered to an adult. In yet another embodiment, the composition includes diphenhydramine in a weight ranging from about 6.25 mg to about 25 mg, per dose, for administration to a child.

Cyproheptadine, or a pharmaceutically acceptable salt or derivative thereof, is also known to be effective as an antihistamine and is suitable for the present invention. Cyproheptadine is generally effective when administered in a dose of at least about 0.05 mg. Particularly in children, effective doses generally range from about 1 mg to about 2 mg, while in adults, effective doses generally range from about 2 mg to about 4 mg. The usual dose in adults is about 12 to about 16 mg per day over 3 to 4 doses. Adults that have previously been treated with cyproheptadine and have developed a tolerance for cyproheptadine, may be administered doses higher than 4 mg. For example, adults with significant tolerances may be given up to about 20 mg per dose without a concern for overdosing and related dangers. Besides its antihistaminic activity, cyproheptadine is mildly sedative and also has anti-serotonin activity. Accordingly, cyproheptadine is useful for treating catamenial migrane headaches and menstrually-related migrane headaches, and for stimulating the appetite. In one embodiment of the invention, the antihistamine is cyproheptadine and is present in an amount ranging from about 0.05 mg to about 20 mg per dose. In another embodiment, cyproheptadine is present in a dose range from about 1 mg to about 4 mg.

Stimulants reduce or alleviate the sedation caused by the antihistamine, as described above. To this end, suitable stimulants include, without limitation, amphetamine class compounds as well as non-amphetamine class compounds, and their pharmaceutically acceptable salts and derivatives, respectively. Examples of suitable amphetamines or “amphetamine-class” compounds include, without limitation, dextromorphan. Examples of suitable non-amphetamine class compounds include, without limitation, caffeine, pemoline, methylphenidate and modafinil.

Caffeine, or a pharmaceutically acceptable salt form thereof, such as caffeine citrate or caffeine sodium benzoate, or a derivative thereof, is known to have effective stimulant activity. Specifically, caffeine's stimulation generally counteracts sleepiness or drowsiness (sedation) and will therefore be effective against the sedation caused by the antihistamine, such as a first-generation antihistamine like diphenhydramine. In addition to the stimulant activity, caffeine has several other pharmacological modes of action in the central nervous system and is capable of providing further effects, such as mild analgesia and analgesic adjuvant properties.

Where caffeine or a pharmaceutical salt or derivative thereof is included in the composition, it should be present in an amount sufficient, per dose, to effectively reduce or alleviate the sedation caused by the antihistamine. To this end, an amount of caffeine citrate of at least about 25 mg, per dose, may generally be effective. More particularly, for a child, two years of age and older, caffeine citrate in a weight range from about 25 mg to about 200 mg, per dose, is generally effective. For an adult, caffeine citrate in the weight range from about 100 mg to about 300 mg, per dose, is generally effective. Compositions including the use of caffeine citrate should not be administered to children under the age of two. In one embodiment of the invention, caffeine, or a pharmaceutically acceptable salt or derivative thereof, is included in a weight of at least about 25 mg per dose. In another embodiment, caffeine is included in a weight ranging from about 50 mg to about 500 mg per dose and, in yet another embodiment, caffeine is included in a weight ranging from about 100 mg to about 300 mg per dose. Similarly, effective amounts or ranges of effective amounts of stimulants other than caffeine, per dose of the composition, are known to and appreciated by those of ordinary skill in the art. Again, the effective dose may vary in accordance to many factors, many of which are described herein with respect to effective amounts of the antihistamine.

Where the stimulant is amphetamine or an amphetamine variant, it should be included in an amount sufficient to at least reduce sedation. Thus, where methylphenidate (Ritalin) and dextroamphetamine are included in the composition, they may be administered in a dosage of as low as about 1 mg and ranging to about 1.25 mg to be effective in a child. Methylphenidate is generally effective in a dosage ranging from about 2.5 mg to about 5 mg for an adult.

In another aspect of the invention, a plurality of stimulants may be included in the present composition. Particularly, multiple amphetamine-class stimulants can be used effectively in combination. For example, the combination of amphetamine salts used in Adderall®, including dextroamphetamine saccrate, amphetamine aspartate, dextroamphetamine sulfate USP, and amphetamine sulfate USP, may be used in equal amounts in the composition to constitute the total stimulant included. More specifically, to provide 10 mg of an amphetamine as the stimulant in a tablet, the tablet may include about 2.5 mg of each of the four forms of amphetamine described for Adderall® above, thereby providing a total amphetamine free-base weight of about 6.3 mg. The particular salts included, and their amounts and ratios, may vary as desired.

Where the antihistamine is not diphenhydramine, or a pharmaceutically acceptable salt thereof, or the stimulant is not caffeine, or a pharmaceutically acceptable salt thereof, the composition of the present invention may further include other active ingredients. For example, the composition may further include sympathomimetic agents, opioids, anti-tussive agents, anti-secretory agents, and combinations thereof. Sympathomimetic agents, such as epinephrine, phenylephrine, ephedrine, psuedoephedrine, and isoproterenol, are useful for the treatment of asthma. Where pseudoephedrine is included, it is typically effective in a dose of about 15 mg for a child, and in a dose ranging from about 30 to about 60 mg for an adult. Opioids, such as hydrocodone, methadone, codeine, codeine derivatives such as hydrocodone, dihydrocodone, and oxycodone, nalbufeine, naloxone, and the like, are useful for analgesia and alleviating pain. Where hydrocodone is included, it is generally effective in a dose ranging form about 2.5 mg to about 5 mg for a child, and a dose ranging from about 5 mg to about 10 mg in an adult. Many opioids, such as dextromethorphan, also serve as anti-tussive agents and are useful as cough suppressing ingredients. Dextromethorphan, where included in the composition, is generally effective in a dose of about 5 mg for a child, and a dose of up to about 60 mg for an adult. Anti-secretory agents include, for example, expectorants such as guaifenesin (Robitussin), and atropinic compounds such as hyoscyamine and scopolamine. Guaifenesin, where included, is generally effective if dosed in an amount of up to about 50 mg for a child, and up to about 200 mg for an adult. Atropine is generally effective in dosages of about 0.04 mg for a child and up to about 0.1 to about 0.2 mg in an adult. Similarly, scopolamine and hyoscyamine are generally effective in dosages ranging from about 0.01 mg to about 0.5 mg depending on whether the patient is a child or an adult. Such agents generally dry mucous membranes and are useful for patients suffering from runny noses, cough, etc.

While the composition of the present invention includes a sedating antihistamine and a sedation-countering stimulant, the present composition is not so limited and may include other non-active components, such as conventionally included excipients. Generally, excipients are non-active ingredients that are useful and/or desirable for formulation purposes, such as to render the composition suitable and/or attractive for consumption and use. For example, with respect to physical properties, ingredients imparting desirable and acceptable hardness, disintegration properties, dissolution rates for releasing therapeutic components, stability, and/or size to effectively deliver the composition may be included. Disintegrants are generally included to facilitate the breakup of a tablet after the tablet is administered to the patient. Examples of disintegrants include, but are not limited to, modified or unmodified starches such as cornstarch, potato starch, wheat starch, or sodium cross-carmelus. With respect to aesthetics, the composition may contain additives that appeal to human senses, such as colorants, fragrances, texture modifiers, and/or flavorants. Suitable colorants include, without limitation, red beet powder, ferric oxide, FD&C dyes, or combinations thereof. Suitable flavoring agents include, for example, fruit flavors or sweeteners such as sodium saccharin, confectioners sugar, sucrose, xylitol, or combinations thereof. It should be understood that these other components should not affect the action or mechanism of action of the active ingredient, and particularly the antihistamine and the stimulant in the composition.

Excipients affecting the properties, mechanisms, and/or rates of release of the antihistamine and/or the stimulant from the composition are beneficial. For example, the composition may be formulated such that release of the actives may be delayed to provide better absorption and better bioavailability for improved therapeutic effect. To this end, enteric coatings or encapsulation-type coatings for enterally administrable compositions are contemplated herein.

The compositions may be formulated for a variety of applications and particularly for enteral and parenteral administration. To this end, the term “enteral” includes administration via a naturally occurring bodily orifice, such as the mouth or the anus. For example, the composition or final formulation may be orally administered and ingested, or rectally administered for effectively delivering the antihistamine and stimulant to the body. Suppositories and the like, capable of dissolving and releasing the actives in the anal cavity for absorption are suitable formulations for rectal administration. In one embodiment of the present invention, a composition including diphenhydramine hydrochloride in a weight ranging from about 25 mg to about 75 mg per dose and caffeine citrate in a weight ranging from about 100 mg to about 300 mg per dose, is formulated for oral or rectal administration to an adult. In another embodiment, a composition including diphenhydramine hydrochloride in a weight ranging from about 6.25 mg to about 25 mg per dose and caffeine citrate in a weight ranging from about 50 mg to about 200 mg per dose, is formulated for oral or rectal administration to a child.

Alternatively, the composition may be formulated for parenteral administration. The term “parenteral”, as used herein with reference to mode of administration, refers to all modes outside the scope of “enteral” administration. The antihistamine and stimulant ranges, described above in the embodiments formulated for enteral administration, are also suitable for parenteral administration. For example, an intravenous formulation of the present composition may include diphenhydramine, in a range from about 25 mg to about 50 mg per dose, and caffeine sodium benzoate, in an amount up to about 500 mg per dose, for administration to a child six years or older, or to an adult.

The formulations may be suitable solids or liquids. Solid formulations include, without limitation, a tablet, a pill, and a capsule such as a softgel or hard-shelled capsule. Orally ingestible solids include, for example, tablets, including chewable tablets, melting tablets (oral dispersables), gelatin tablets, hard and soft gel-caps, and the like. Liquid formulations are also generally orally ingestible and include, for example, suspensions, syrups, and the like. Tablets and pills may advantageously be small and convenient to swallow, and have a generally accepted taste and appearance to promote ingestion. The formulations may be prepared by processes known in the art of pharmaceutical manufacturing. For example, tablets may be formed either by direct compression of the components or by granulation of the components followed by the compression. Additional ingredients may be included during compression where desired. For example, the granular mixture may contain one or more lubricants to inhibit sticking during compression. Examples of suitable lubricants include, but are not limited to, stearic acid, palmetto stearate, talc, oils, and the like. Advantageously, the composition of the present invention is formulated into a chewable tablet, a tablet that melts in the mouth or under the tongue upon exposure to saliva, and/or an enteric coated tablet. In addition, capsules may be prepared by blending the antihistamine(s) and stimulant(s) with desirable excipients and filling the capsule with the blended mixture using conventional filling equipment. Gelatin capsule, and in particular, soft-shelled gelatin capsules are increasingly popular. Further, where desired, the capsule may be coated for added benefits.

The pharmaceutical compositions of the present invention may be administered to a patient for treating a variety of histamine-mediated symptoms. For example, the compositions may be administered for alleviating viral and allergic rhinitis, and other allergy symptoms in the patient. Depending upon the patient, the composition may be administered in an amount having an amount of the antihistamine sufficient to alleviate congestion, cough, pain, itching, swelling, and the like, and an amount of the stimulant sufficient to at least alleviate the patient's sedation caused by the antihistamine. The composition may also be administered to induce a particular effect, such as an anticholinergic effect, an analgesic effect, an analgesic adjuvant, a soporific effect, an anti-secretory effect by drying or dehydrating mucous membranes, and/or a combination effect thereof. Many of these effects are induced by the particular antihistamine and/or stimulant included in the composition. The patient in need of treatment may be a child or an adult suffering from the symptom(s) for which the composition is administered. Amounts of the composition administered will generally depend upon the particular formulation of the composition and the concentration of the antihistamine(s) and/or stimulant(s) in the formulation. The amount administered will also depend upon various factors related to the physical make-up of the patient. For example, age, health, weight, prior medical history, extent and degree of symptoms, and overall medical diagnosis are a few of the factors, appreciated by persons of ordinary skill in the art, that should be taken into consideration prior to administration of the composition.

Thus, the present invention provides compositions, and methods of use thereof, for treating histamine-mediated responses and, in particular, for treating allergies and related undesirable and discomforting symptoms associated with allergies. To this end, the sedating effects of more potent, therapeutically effective antihistamines, such as diphenhydramine, are countered by the inclusion of one or more stimulants in the composition. Thus, potent, sedative antihistamines, which have been overlooked and excluded in favor of less effective, non-sedating antihistamines for providing antihistaminic effects, may once again be safely administered in effective doses with reduced or minimal accompanying sedation. Inclusion of more potent antihistamines translates into smaller dosages per administration and, therefore, less costly pharmaceutical compositions and formulations. As such, the present compositions may more likely to be taken by the patient. Further, the overall size and amount of ingredients in the composition may be smaller as many antihistamines provide additional physiological effects thereby eliminating the need for additional or separate ingredients to provide the same effect(s). Fewer ingredients and smaller dosages render the compositions of the present invention less costly in terms of research, development and government approval.

While the present invention has been illustrated by the description of embodiments thereof, and while the embodiments have been described in considerable detail, it is not intended to restrict or in any way limit the scope of the appended claims to such detail. Additional advantages and modifications will be readily apparent to those skilled in the art. The invention in its broader aspects is therefore not limited to the specific details, representative apparatus and method, and illustrated examples described. Accordingly, departures may be made from such details without departing from the scope or spirit of Applicant's general inventive concept.

What is claimed is:

1. A pharmaceutical composition comprising a sedating antihistamine in an amount sufficient to cause sedation and a stimulant in an amount sufficient to alleviate the sedation caused by the antihistamine, wherein the composition is in a pharmaceutically acceptable dose formulation and excludes caffeine or a pharmaceutically acceptable salt or derivative thereof.

2. The composition of claim 1 wherein the antihistamine comprises at least one of diphenhydramine, cyproheptadine hydrochloride, brompheneramine, hydroxyzine, chloropheniramine, pyrilamine maleate, pyrilamine tannate, acepromazine, aceprometazine, alimemazine, alimemazine tartrate, amoxydramine camsilate, antazoline chlorhydrate, antazoline mesilate, antazoline phosphate, astemizole, azatadine dimaleate, azelastine hydrochloride, bamipine hydrochloride, benactyzine hydrochloride, bretylium tosilate, bromazine hydrochloride, brompheniramine maleate, buclizine dihydrochloride, bufexamac, carbinoxamine maleate acid, cetiedil citrate, cetirizine dihydrochloride, chlorcyclizine hydrochloride, chlorphenamine maleate, chlorphenoxamine hydrochloride, chlorprothixene hydrochloride, cinnarizine, clemastine fumarate, clemizole hexachlorophenate, clemizole penicilline, clemizole undecylenate, clocinizine dihydrochloride, clofedanol, clofenetamine hydrochloride, cyclizine hydrochloride, dexchlorpheniramine maleate, di(acefylline)diphenhydramine, difencloxazine, dimelazine hydrochloride, dimenhydrinate, dimethoxanate hydrochloride, cimetotiazine mesilate, diphenhydramine hydrochloride, diphenhydramine mesilate, diphenylpyraline hydrochloride, diproqualone camsilate, dixyrazine, doxylamine succinate, eprozinol dihydrochloride, etodroxizine dimaleate, etybenzatropine bromhydrate, etybenzatropine hydrochloride, etymemazine hydrochloride, fenethazine hydrochloride, fenoxazoline hydrochloride, fenpentadiol, flunarizine hydrochloride, flupentixol decanoate, flupentixol dihydrochloride, histapyrrodine hydrochloride, hydroxyzine dihydrochloride, hydroxyzine embonate, indoramine hydrochloride, isothipendyl hydrochloride, ketotifene fumarate, levocabastine hydrochloride, levomepromazine, levomepromazine hydrochloride, levomepromazine embonate, levomepromazine maleate, loratadine, maprotiline hydrochloride, maprotiline mesilate, maprotiline resinate, meclozine hydrochloride, mecysteine hydrochloride, medifoxamine fumarate, mefenidramium metilsulfate, mepyramine maleate, mequitazine, methaqualone, methdilazine hydrochloride, metixene hydrochloride, mizolastine, moxisylyte hydrochloride, niaprazine, orphenadrine hydrochloride, oxaflumazine disuccinate, oxatomide, oxolamine benzilate, oxolamine citrate, oxomemazine, oxomemazine hydrochloride, parathiazine teoclate, perimetazine, pheniramine maleate, phenoxybenzamine hydrochloride, phenyltoloxamine, phenyltoloxamine citrate, pimethixene, pipotiazine, pipretecol dihydrochloride, pizotifene malate, prednazoline, profenamine hydrochloride, promethazine, promethazine hydrochloride, promethazine embonate, promethazine polyvinylbenzene-metacrylate, propiomazine, terfenadine, thenalidine tartrate, thenyldiamine hydrochloride, thiazinamium metilsulfate, tripelennamine hydrochloride, triprolidine hydrochloride, and tymazoline hydrochloride.

3. The composition of claim 1 wherein the antihistamine is diphenhydramine or a pharmaceutically acceptable salt or derivative thereof and present in a weight of at least about 6 mg per dose of the composition.

4. The composition of claim 1 wherein the antihistamine is diphenhydramine or a pharmaceutically acceptable salt or derivative thereof and present in a weight range from about 6 mg to about 100 mg per dose of the composition.

5. The composition of claim 1 wherein the antihistamine is cyproheptadine or a pharmaceutically acceptable salt or derivative thereof and present in a weight range from about 0.5 mg to about 20 per dose of the composition.

6. The composition of claim 1 wherein the antihistamine is cyproheptadine or a pharmaceutically acceptable salt or derivative thereof and present in a weight range from about 1 mg to about 4 mg per dose of the composition.

7. The composition of claim 1 wherein the stimulant is at least one amphetamine or a pharmaceutically acceptable salt or derivative thereof.

8. The composition of claim 1 wherein the stimulant is a combination of dextroamphetamine saccarate, amphetamine aspartate, dextroamphetamine sulfate, and amphetamine sulfate wherein each amphetamine is present in a weight of at least about 1.25 mg per dose of the composition.

9. The composition of claim 1 wherein the stimulant comprises at least one of dextroamphetamine, pemoline, methylphenidate, modafinil, and pharmaceutically acceptable salts or derivatives thereof.

10. The composition of claim 1 further comprising at least one active selected from a sympathomimetic agent, an opioid agent, an anti-tussive agent, and an anti-secretory agent.

11. The composition of claim 1 wherein the formulation is adapted for at least one of enteral and parenteral administration.

12. A pharmaceutical composition comprising a sedating antihistamine in an amount sufficient to cause sedation and a stimulant in an amount sufficient to alleviate the sedation caused by the antihistamine, wherein the composition is in a pharmaceutically acceptable dose formulation and excludes diphenhydramine or a pharmaceutically acceptable salt or derivative thereof.

13. The composition of claim 12 wherein the antihistamine comprises at least one of cyproheptadine hydrochloride, brompheneramine, hydroxyzine, chloropheniramine, pyrilamine maleate, pyrilamine tannate, acepromazine, aceprometazine, alimemazine, alimemazine tartrate, amoxydramine camsilate, antazoline chlorhydrate, antazoline mesilate, antazoline phosphate, astemizole, azatadine dimaleate, azelastine hydrochloride, bamipine hydrochloride, benactyzine hydrochloride, bretylium tosilate, bromazine hydrochloride, brompheniramine maleate, buclizine dihydrochloride, bufexamac, carbinoxamine maleate acid, cetiedil citrate, cetirizine dihydrochloride, chlorcyclizine hydrochloride, chlorphenamine maleate, chlorphenoxamine hydrochloride, chlorprothixene hydrochloride, cinnarizine, clemastine fumarate, clemizole hexachlorophenate, clemizole penicilline, clemizole undecylenate, clocinizine dihydrochloride, clofedanol, clofenetamine hydrochloride, cyclizine hydrochloride, dexchlorpheniramine maleate, difencloxazine, dimelazine hydrochloride, dimenhydrinate, dimethoxanate hydrochloride, cimetotiazine mesilate, diphenylpyraline hydrochloride, diproqualone camsilate, dixyrazine, doxylamine succinate, eprozinol dihydrochloride, etodroxizine dimaleate, etybenzatropine bromhydrate, etybenzatropine hydrochloride, etymemazine hydrochloride, fenethazine hydrochloride, fenoxazoline hydrochloride, fenpentadiol, flunarizine hydrochloride, flupentixol decanoate, flupentixol dihydrochloride, histapyrrodine hydrochloride, hydroxyzine dihydrochloride, hydroxyzine embonate, indoramine hydrochloride, isothipendyl hydrochloride, ketotifene fumarate, levocabastine hydrochloride, levomepromazine, levomepromazine hydrochloride, levomepromazine embonate, levomepromazine maleate, loratadine, maprotiline hydrochloride, maprotiline mesilate, maprotiline resinate, meclozine hydrochloride, mecysteine hydrochloride, medifoxamine fumarate, mefenidramium metilsulfate, mepyramine maleate, mequitazine, methaqualone, methdilazine hydrochloride, metixene hydrochloride, mizolastine, moxisylyte hydrochloride, niaprazine, orphenadrine hydrochloride, oxaflumazine disuccinate, oxatomide, oxolamine benzilate, oxolamine citrate, oxomemazine, oxomemazine hydrochloride, parathiazine teoclate, perimetazine, pheniramine maleate, phenoxybenzamine hydrochloride, phenyltoloxamine, phenyltoloxamine citrate, pimethixene, pipotiazine, pipretecol dihydrochloride, pizotifene malate, prednazoline, profenamine hydrochloride, promethazine, promethazine hydrochloride, promethazine embonate, promethazine polyvinylbenzene-metacrylate, propiomazine, terfenadine, thenalidine tartrate, thenyldiamine hydrochloride, thiazinamium metilsulfate, tripelennamine hydrochloride, triprolidine hydrochloride, and tymazoline hydrochloride.

14. The composition of claim 12 wherein the antihistamine is cyproheptadine or a pharmaceutically acceptable salt or derivative thereof and present in a weight range from about 0.5 mg to about 20 per dose of the composition.

15. The composition of claim 12 wherein the antihistamine is cyproheptadine or a pharmaceutically acceptable salt or derivative thereof and present in a weight range from about 1 mg to about 4 mg per dose of the composition.

16. The composition of claim 12 wherein the stimulant is caffeine and present in a weight range from about 25 mg to about 500 mg per dose of the composition.

17. The composition of claim 12 wherein the stimulant is caffeine and present in a weight range from about 100 mg to about 300 mg per dose of the composition.

18. The composition of claim 12 wherein the stimulant is at least one amphetamine or a pharmaceutically acceptable salt or derivative thereof.

19. The composition of claim 12 wherein the stimulant is a combination of dextroamphetamine saccarate, amphetamine aspartate, dextroamphetamine sulfate, and amphetamine sulfate wherein each amphetamine is present in a weight of at least about 1.25 mg per dose of the composition.

20. The composition of claim 12 wherein the stimulant comprises at least one of dextroamphetamine, pemoline, methylphenidate, modafinil, and pharmaceutically acceptable salts or derivatives thereof.

21. The composition of claim 12 further comprising at least one active selected from a sympathomimetic agent, an opioid agent, an anti-tussive agent, and an anti-secretory agent.

22. The composition of claim 12 in a formulation adapted for at least one of enteral and parenteral administration.

23. A pharmaceutical composition consisting essentially of at least one sedating antihistamine in an amount sufficient to cause sedation and at least one stimulant in an amount sufficient to alleviate the sedation caused by the antihistamine, the composition in a pharmaceutically acceptable dose formulation.

24. The composition of claim 23 wherein the antihistamine is diphenhydramine or a pharmaceutically acceptable salt or derivative thereof and present in a weight of at least about 6 mg per dose of the composition.

25. The composition of claim 23 wherein the antihistamine is diphenhydramine or a pharmaceutically acceptable salt or derivative thereof and present in a weight range from about 6 mg to about 100 mg per dose of the composition.

26. The composition of claim 23 wherein the antihistamine is cyproheptidine or a pharmaceutically acceptable salt or derivative thereof and present in a weight range from about 0.5 mg to about 20 mg per dose of the composition.

27. The composition of claim 23 wherein the antihistamine is cyproheptadine or a pharmaceutically acceptable salt or derivative thereof and present in a weight range from about 1 mg to about 4 mg per dose of the composition.

28. The composition of claim 23 wherein the stimulant is caffeine or a pharmaceutically acceptable salt or derivative thereof and present in a weight of range from about 25 mg to about 500 mg per dose of the composition.

29. The composition of claim 23 wherein the stimulant is caffeine or a pharmaceutically acceptable salt or derivative thereof and present in a weight range from about 100 mg to about 300 mg per dose of the composition.

30. The composition of claim 23 wherein the stimulant is at least one amphetamine or a pharmaceutically acceptable salt or derivative thereof.

31. The composition of claim 23 wherein the stimulant is a combination of dextroamphetamine saccarate, amphetamine aspartate, dextroamphetamine sulfate, and amphetamine sulfate wherein each amphetamine is present in a weight of at least about 1.25 mg per dose of the composition.

32. The composition of claim 23 wherein the stimulant is at least one consisting of dextroamphetamine, pemoline, methylphenidate, modafinil, and pharmaceutically acceptable salts or derivatives thereof.

33. The composition of claim 23 in a formulation adapted for at least one of enteral and parenteral administration.

34. The composition of claim 23 in a formulation adapted for at least one of enteral and parenteral administration, wherein
the antihistamine is diphenhydramine or a pharmaceutically acceptable salt or derivative thereof and present in a weight range from about 6 mg to about 100 mg per dose of the composition; and
the stimulant is caffeine or a pharmaceutically acceptable salt or derivative thereof and present in a weight range from about 25 mg to about 500 mg per dose of the composition.

35. The composition of claim 23 in a formulation adapted for at least one of enteral and parenteral administration, wherein
the antihistamine is cyproheptadine or a pharmaceutically acceptable salt or derivative thereof and present in a weight range from about 0.5 mg to about 20 mg per dose of the composition; and
the stimulant is caffeine or a pharmaceutically acceptable salt or derivative thereof and present in a weight range from about 25 mg to about 500 mg per dose of the composition.

36. The composition of claim 23 in a formulation adapted for at least one of enteral and parenteral administration, wherein
the antihistamine is diphenhydramine or a pharmaceutically acceptable salt or derivative thereof and present in a weight range from about 6 mg to about 100 mg per dose of the composition; and
the stimulant is at least one of amphetamine, dextroamphetamine, pemoline, methylphenidate, modafinil, and pharmaceutically acceptable salts or derivatives thereof.

37. The composition of claim 23 in a formulation adapted for at least one of enteral and parenteral administration, wherein
the antihistamine is cyproheptadine or a pharmaceutically acceptable salt or derivative thereof and present in a weight range from about 0.5 mg to about 20 mg per dose of the composition; and
the stimulant is at least one of amphetamine, dextroamphetamine, pemoline, methylphenidate, modafinil, and pharmaceutically acceptable salts or derivatives thereof.

38. A pharmaceutical composition consisting essentially of:
diphenhydramine or a pharmaceutically acceptable salt or derivative thereof and present in a weight range from about 6 mg to about 100 mg per dose of the composition; and
caffeine or a pharmaceutically acceptable salt or derivative thereof and present in a weight range from about 25 mg to about 500 mg per dose of the composition,
the composition in a pharmaceutically acceptable dose formulation.

39. A pharmaceutical composition consisting essentially of:
cyproheptadine or a pharmaceutically acceptable salt or derivative thereof in a weight range from about 0.5 mg to about 20 mg per dose of the composition; and
caffeine or a pharmaceutically acceptable salt or derivative thereof and present in a weight range from about 25 mg to about 500 mg per dose of the composition.

40. A method of treating a histamine-mediated response in a patient, the method comprising:
directing administration of a pharmaceutical composition comprising at least one sedating antihistamine and at least one stimulant to the patient, the composition excluding caffeine or a pharmaceutically acceptable salt or derivative thereof and administered in a dose sufficient for the antihistamine to treat the histamine-mediated response while causing sedation in the patient and the stimulant to alleviate the sedation caused by the antihistamine.

41. The method of claim 40 further comprising administering the composition to the patient as directed.

42. The method of claim 40 further comprising formulating the composition into a pharmaceutically acceptable formulation for at least one of enteral and parenteral administration.

43. The method of claim 40 further comprising formulating the composition into one of a pill, a soft-shell gelatin capsule, a hard-shell gelatin capsule, a gel-tab, a chewable tablet, and an oral-dispersable tablet.

44. The method of claim 40 wherein the antihistamine comprises at least one of diphenhydramine, cyproheptadine hydrochloride, brompheneramine, hydroxyzine, chloropheniramine, pyrilamine maleate, pyrilamine tannate, acepromazine, aceprometazine, alimemazine, alimemazine tartrate, amoxydramine camsilate, antazoline chlorhydrate, antazoline mesilate, antazoline phosphate, astemizole, azatadine dimaleate, azelastine hydrochloride, bamipine hydrochloride, benactyzine hydrochloride, bretylium tosilate, bromazine hydrochloride, brompheniramine maleate, buclizine dihydrochloride, bufexamac, carbinoxamine maleate acid, cetiedil citrate, cetirizine dihydrochloride, chlorcyclizine hydrochloride, chlorphenamine maleate, chlorphenoxamine hydrochloride, chlorprothixene hydrochloride, cinnarizine, clemastine fumarate, clemizole hexachlorophenate, clemizole penicilline, clemizole undecylenate, clocinizine dihydrochloride, clofedanol, clofenetamine hydrochloride, cyclizine hydrochloride, dexchlorpheniramine maleate, di(acefylline)diphenhydramine, difencloxazine, dimelazine hydrochloride, dimenhydrinate, dimethoxanate hydrochloride, cimetotiazine mesilate, diphenhydramine hydrochloride, diphenhydramine mesilate, diphenylpyraline hydrochloride, diproqualone camsilate, dixyrazine, doxylamine succinate, eprozinol dihydrochloride, etodroxizine dimaleate, etybenzatropine bromhydrate, etybenzatropine hydrochloride, etymemazine hydrochloride, fenethazine hydrochloride, fenoxazoline hydrochloride, fenpentadiol, flunarizine hydrochloride, flupentixol decanoate, flupentixol dihydrochloride, histapyrrodine hydrochloride, hydroxyzine dihydrochloride, hydroxyzine embonate, indoramine hydrochloride, isothipendyl hydrochloride, ketotifene fumarate, levocabastine hydrochloride, levomepromazine, levomepromazine hydrochloride, levomepromazine embonate, levomepromazine maleate, loratadine, maprotiline hydrochloride, maprotiline mesilate, maprotiline resinate, meclozine hydrochloride, mecysteine hydrochloride, medifoxamine fumarate, mefenidramium metilsulfate, mepyramine maleate, mequitazine, methaqualone, methdilazine hydrochloride, metixene hydrochloride, mizolastine, moxisylyte hydrochloride, niaprazine, orphenadrine hydrochloride, oxaflumazine disuccinate, oxatomide, oxolamine benzilate, oxolamine citrate, oxomemazine, oxomemazine hydrochloride, parathiazine teoclate, perimetazine, pheniramine maleate, phenoxybenzamine hydrochloride, phenyltoloxamine, phenyltoloxamine citrate, pimethixene, pipotiazine, pipretecol dihydrochloride, pizotifene malate, prednazoline, profenamine hydrochloride, promethazine, promethazine hydrochloride, promethazine embonate, promethazine polyvinylbenzene-metacrylate, propiomazine, terfenadine, thenalidine tartrate, thenyldiamine hydrochloride, thiazinamium metilsulfate, tripelennamine hydrochloride, triprolidine hydrochloride, and tymazoline hydrochloride.

45. The method of claim 40 wherein the stimulant comprises at least one amphetamine or a pharmaceutically acceptable salt or derivative thereof.

46. The method of claim 40 wherein the stimulant comprises at least one of dextroamphetamine, pemoline, methylphenidate, modafinil, and a pharmaceutically acceptable salt or derivative thereof.

47. The method of claim 40 wherein the dose directed for administration comprises diphenhydramine or a pharmaceutically acceptable salt or derivative thereof as the antihistamine in an amount of at least about 6 mg.

48. The method of claim 40 wherein the dose directed for administration comprises cyproheptadine or a pharmaceutically acceptable salt or derivative thereof as the antihistamine in an amount ranging from about 0.5 mg to about 20 mg.

49. The method of claim 40 wherein the dose directed for administration further comprises at least one active selected from a sympathomimetic agent, an opioid agent, an anti-tussive agent, and an anti-secretory agent.

50. The method of claim 40 further comprising administering the composition to the patient to provide at least one effect selected from an anti-cholinergic effect, an analgesic effect, an analgesic adjuvant effect, a soporific effect, an anti-secretory effect, and an appetite stimulant effect, in the patient.

51. A method of treating a histamine-mediated response in a patient, the method comprising:
directing administration of a pharmaceutical composition comprising at least one sedating antihistamine and at least one stimulant to the patient, the composition excluding diphenhydramine or a pharmaceutically acceptable salt or derivative thereof and administered in a dose sufficient for the antihistamine to treat the histamine-mediated response while causing sedation in the patient and the stimulant to alleviate the sedation caused by the antihistamine.

52. The method of claim 51 further comprising administering the composition to the patient as directed.

53. The method of claim 51 further comprising formulating the composition into a pharmaceutically acceptable formulation for at least one of enteral and parenteral administration.

54. The method of claim 51 further comprising formulating the composition into one of a pill, a soft-shell gelatin capsule, a hard-shell gelatin capsule, a gel-tab, a chewable tablet, and an oral-dispersable tablet.

55. The method of claim 51 wherein the antihistamine comprises at least one of cyproheptadine hydrochloride, brompheneramine, hydroxyzine, chloropheniramine, pyrilamine maleate, pyrilamine tannate, acepromazine, aceprometazine, alimemazine, alimemazine tartrate, amoxydramine camsilate, antazoline chlorhydrate, antazoline mesilate, antazoline phosphate, astemizole, azatadine dimaleate, azelastine hydrochloride, bamipine hydrochloride, benactyzine hydrochloride, bretylium tosilate, bromazine hydrochloride, brompheniramine maleate, buclizine dihydrochloride, bufexamac, carbinoxamine maleate acid, cetiedil citrate, cetirizine dihydrochloride, chlorcyclizine hydrochloride, chlorphenamine maleate, chlorphenoxamine hydrochloride, chlorprothixene hydrochloride, cinnarizine, clemastine fumarate, clemizole hexachlorophenate, clemizole penicilline, clemizole undecylenate, clocinizine dihydrochloride, clofedanol, clofenetamine hydrochloride, cyclizine hydrochloride, dexchlorpheniramine maleate, difencloxazine, dimelazine hydrochloride, dimenhydrinate, dimethoxanate hydrochloride, cimetotiazine mesilate, diphenylpyraline hydrochloride, diproqualone camsilate, dixyrazine, doxylamine succinate, eprozinol dihydrochloride, etodroxizine dimaleate, etybenzatropine bromhydrate, etybenzatropine hydrochloride, etymemazine hydrochloride, fenethazine hydrochloride, fenoxazoline hydrochloride, fenpentadiol, flunarizine hydrochloride, flupentixol decanoate, flupentixol dihydrochloride, histapyrrodine hydrochloride, hydroxyzine dihydrochloride, hydroxyzine embonate, indoramine hydrochloride, isothipendyl hydrochloride, ketotifene fumarate, levocabastine hydrochloride, levomepromazine, levomepromazine hydrochloride, levomepromazine embonate, levomepromazine maleate, loratadine, maprotiline hydrochloride, maprotiline mesilate, maprotiline resinate, meclozine hydrochloride, mecysteine hydrochloride, medifoxamine fumarate, mefenidramium metilsulfate, mepyramine maleate, mequitazine, methaqualone, methdilazine hydrochloride, metixene hydrochloride, mizolastine, moxisylyte hydrochloride, niaprazine, orphenadrine hydrochloride, oxaflumazine disuccinate, oxatomide, oxolamine benzilate, oxolamine citrate, oxomemazine, oxomemazine hydrochloride, parathiazine teoclate, perimetazine, pheniramine maleate, phenoxybenzamine hydrochloride, phenyltoloxamine, phenyltoloxamine citrate, pimethixene, pipotiazine, pipretecol dihydrochloride, pizotifene malate, prednazoline, profenamine hydrochloride, promethazine, promethazine hydrochloride, promethazine embonate, promethazine polyvinylbenzene-metacrylate, propiomazine, terfenadine, thenalidine tartrate, thenyldiamine hydrochloride, thiazinamium metilsulfate, tripelennamine hydrochloride, triprolidine hydrochloride, and tymazoline hydrochloride.

56. The method of claim 51 wherein the stimulant comprises at least one amphetamine or a pharmaceutically acceptable salt or derivative thereof.

57. The method of claim 51 wherein the stimulant comprises at least one of dextroamphetamine, pemoline, methylphenidate, modafinil, and a pharmaceutically acceptable salt or derivative thereof.

58. The method of claim 51 wherein the dose directed for administration comprises caffeine or a pharmaceutically acceptable salt or derivative thereof as the antihistamine in an amount ranging from about 25 mg to about 500 mg.

59. The method of claim 51 wherein the dose directed for administration comprises a combination of dextroamphetamine saccarate, amphetamine aspartate, dextroamphetamine sulfate, and amphetamine sulfate as the stimulant with each amphetamine present in a weight of at least about 1.25 mg.

60. The method of claim 51 wherein the dose directed for administration further comprises at least one active selected from a sympathomimetic agent, an opioid agent, an anti-tussive agent, and an anti-secretory agent.

61. The method of claim 51 further comprising administering the composition to the patient to provide at least one effect selected from an anti-cholinergic effect, an analgesic effect, an analgesic adjuvant effect, a soporific effect, an anti-secretory effect, and an appetite stimulant effect, in the patient.

62. A method of treating a histamine-mediated response in a patient, the method comprising directing administration of a pharmaceutical composition consisting essentially of a sedating antihistamine and a stimulant to the patient, the composition administered as a pharmaceutically acceptable dose formulation and in an amount sufficient for the antihistamine to treat the histamine-mediated response while causing sedation in the patient and the stimulant to alleviate the sedation caused by the antihistamine.

63. The method of claim 62 further comprising administering the composition to the patient as directed.

64. The method of claim 62 further comprising formulating the composition into a pharmaceutically acceptable formulation for at least one of enteral and parenteral administration.

65. The method of claim 62 further comprising formulating the composition into one of a pill, a soft-shell gelatin capsule, a hard-shell gelatin capsule, a gel-tab, a chewable tablet, and an oral-dispersable tablet.

66. The method of claim 62 wherein the stimulant comprises at least one amphetamine or a pharmaceutically acceptable salt or derivative thereof.

67. The method of claim 62 wherein the stimulant comprises at least one of dextroamphetamine, pemoline, methylphenidate, modafinil, and a pharmaceutically acceptable salt or derivative thereof.

68. The method of claim 62 wherein the dose directed for administration comprises diphenhydramine or a pharmaceutically acceptable salt or derivative thereof as the antihistamine in an amount of at least about 6 mg and caffeine or a pharmaceutically acceptable salt or derivative thereof as the stimulant in an amount ranging from about 25 mg to about 500 mg.

69. The method of claim 62 wherein the dose directed for administration comprises cyproheptadine or a pharmaceutically acceptable salt or derivative thereof as the antihistamine in an amount ranging from about 0.5 mg to about 20 mg and caffeine or a pharmaceutically acceptable salt or derivative thereof as the stimulant in an amount ranging from about 25 mg to about 500 mg.

70. The method of claim 62 wherein the dose directed for administration comprises diphenhydramine or a pharmaceutically acceptable salt or derivative thereof as the antihistamine in an amount of at least about 6 mg and a combination of dextroamphetamine saccarate, amphetamine aspartate, dextroamphetamine sulfate, and amphetamine sulfate as the stimulant with each amphetamine present in a weight of at least about 1.25 mg.

71. The method of claim 62 wherein the dose directed for administration comprises cyproheptadine or a pharmaceutically acceptable salt or derivative thereof as the antihistamine in an amount ranging from about 0.5 mg to about 20 mg and a combination of dextroamphetamine saccarate, amphetamine aspartate, dextroamphetamine sulfate, and amphetamine sulfate as the stimulant with each amphetamine present in a weight of at least about 1.25 mg.

72. The method of claim 62 further comprising administering the composition to the patient to provide at least one effect selected from an anti-cholinergic effect, an analgesic effect, an analgesic adjuvant effect, a soporific effect, an anti-secretory effect, and an appetite stimulant effect, in the patient.

73. The method of claim 62 wherein the dose directed for administration further comprises at least one active selected from a sympathomimetic agent, an opioid agent, an anti-tussive agent, and an anti-secretory agent.

74. A method of treating a histamine-mediated response in a child, the method comprising directing administration of a pharmaceutical composition consisting essentially of a sedating antihistamine selected from diphenhydramine, cyproheptadine, and a pharmaceutically acceptable salt or derivative thereof, and a stimulant selected from caffeine, dextroamphetamine, pemoline, methylphenidate, modafinil, and a pharmaceutically acceptable salt or derivative thereof, to the patient, the composition administered as a pharmaceutically acceptable dose formulation and in an amount sufficient for the antihistamine to treat the histamine-mediated response while causing sedation in the patient and the stimulant to alleviate the sedation caused by the antihistamine.
